(19) United States
(12) Patent Application Publication
(10) Pub. No.: US 2010/0326432 A1
(43) Pub. Date: Dec. 30, 2010

(54) METERED DOSE DISPENSER

(76) Inventors: Andrew D. Child, Lancashire (GB); Stephen D. Helm, Lancashire (GB)

Correspondence Address: 3M INNOVATIVE PROPERTIES COMPANY, PO BOX 33427, ST. PAUL, MN, 55133-3427, US

(21) Appl. No.: 12/681,297

(22) PCT Filed: Oct. 1, 2008
(86) PCT No.: PCT/US08/78406

§ 371 Date: Sep. 2, 2010

(30) Foreign Application Priority Data
Oct. 4, 2007 (GB) 0719257.8

(51) Int. Cl. A61M 11/00

(52) U.S. Cl. 128/200.23

(57) ABSTRACT

A metered dose inhaler containing a medicinal formulation of medicament, HFA 134a and/or HFA227, and being substantially free of ethanol and surfactant, with a metering valve comprising a helical spring, a seal, a seal support and a sliding valve stem, wherein the valve is configured and arranged such that a region of compressive contact is defined where a surface applying force to the seal is substantially flat and extends in an arc through an angle in the range from about 180 to about 360 degrees.

### FIELD OF THE INVENTION

The present invention relates to metered dose dispensers, in particular pressurized metered dose inhalers, as well as to metered aerosol valves for use with such dispensers.

### BACKGROUND

Pressurized metered dose dispensers have been known for over 50 years, particularly inhalers for the treatment of asthma, but also for other diseases where the lung, throat or nasal passages are suitable sites for delivery of drugs. A formulation of drug in such a dispenser is typically either in the form of a suspension or a solution in propellant system, depending on the solubility of the drug in the formulation. Most often an excipient, such as surfactant and/or ethanol have been added to the aerosol formulation to facilitate the provision of a stable formulation, e.g. ethanol in a solution formulation to ensure that the drug remains dissolved or a surfactant in suspension formulation to aid in dispersion of the drug particles. Recently the CFC propellants used in pressurized metered dose dispensers have been replaced with HFA 134a and/or HFA 227. And more recently in an effort to avoid any potential undesirable interaction with auxiliary components of medicinal aerosol formulations, some medicinal aerosol formulations based on HFA 134a and/or HFA 227 are substantially free of surfactant (e.g. less than 0.0001 wt % with respect to drug) and/or substantially free of ethanol (e.g. less than 0.1 wt % with respect to the formulation) or they are free of surfactant and/or free of ethanol.

Metered dose valves dispense drug formulation volumetrically to dispense accurate weights of drug to a patient. They typically comprise a metering chamber located between an inner seal (metering seal) and an outer seal (diaphragm seal) and a valve stem passing through the metering chamber in sliding sealing engagement with the two seals. In such valves, generally, the valve stem is biased (typically outwardly) in its rest position through a spring and the valve stem is displaced (typically depressed inwardly) to an actuation or firing position to dispense a dose, and following actuation the valve stem is release allowing it to return to its rest position. There are many designs for metered dose valves. Two such designs are disclosed in GB 1336379 and GB 2077229. Metered dose valves in accordance with those described in these documents have been on the market for over 30 years.

### SUMMARY OF THE INVENTION

Unexpectedly it has been found that with dispensers fitted with such a commercial valve containing a medicinal formulation the formulation comprising a medicament and HFA 134a and/or HFA 227 and being substantially free of surfactant and substantially free of ethanol, can show over lifetime of the device a significant and continuous drop in return force (force acting on the valve stem during its return back to its rest position after actuation) and sometimes a drop in return force to such an extent that the valve stem does not return. This is particularly surprising since such an effect has not been previously observed in the 30 plus years such valves have been marketed. Although the underlying cause of this effect is not clearly understood, it seems that the effect may be somehow associated with valve design where the spring operates against a seal, and surprisingly the effect can be effectively reduced by configuring and arranging the valve such that a region of compressive contact is defined where a surface applying said force to the seal is substantially flat and extends in an arc through an angle in the range from about 180 to about 360 degrees.

Accordingly, the invention provides a metered dose inhaler comprising an aerosol container fitted with a metered dose aerosol valve and containing a medicinal formulation, the formulation comprising a medicament and HFA 134a and/or HFA 227 and being substantially free of ethanol and substantially free of surfactant, and the valve comprising a helical spring, a seal, a seal support and a valve stem, the valve stem and seal being in mutual sliding sealing engagement, the helical spring comprising a coil of elongate material and exerting a force compressing the seal against the seal support, wherein the valve is configured and arranged such that a region of compressive contact is defined where a surface applying said force to the seal is substantially flat and extends in an arc through an angle in the range from about 180 to about 360 degrees.

In preferred embodiments, the end portion of the helical spring facing the seal has a substantially flat surface extending from the corresponding terminus of the coil of the elongate material in an arc through an angle in the range from about 180 to about 360 degrees.

In alternative embodiments, a split-washer extending in an arc through an angle in the range from about 270 up to but not including 360 degrees or a washer may be positioned between the helical spring and the seal. In connection with such embodiments it is noted that a washer is mentioned in a document published in 1968, GB 1136886, however the function of the washer is not disclosed, nor any details of the aerosol formulation which at the time of publication GB 113886 were based on CFC propellants containing appreciable amounts of ethanol or surfactant.

In another aspect the invention provides a metered dose aerosol valve comprising a helical spring, a seal, a seal support and a valve stem, the valve stem and seal being in mutual sliding sealing engagement, the helical spring comprising a coil of elongate material and exerting a force compressing the seal against the seal support, wherein the helical spring has an end portion facing the seal, said end portion of the helical spring having a substantially flat surface extending from the corresponding terminus of the coil of the elongate material in an arc through an angle in the range from about 180 to about 360 degrees

### BRIEF DESCRIPTION OF THE DRAWINGS

FIG. 1 shows a partial cross-sectional view of an exemplary metered dose dispensing valve in accordance with the invention.

FIG. 2a shows an isometric view of an exemplary compression spring for use in a metered dose dispensing valve in accordance with the invention, in particular the compression spring used in the valve of FIG. 1.

FIG. 2b shows an isometric view of an alternative exemplary compression spring for use in a metered dose dispensing valve in accordance with the invention.

FIG. 3 shows a partial cross-sectional view of another exemplary metered dose dispensing valve for use in a metered dose inhaler in accordance with the invention.

FIG. 4 shows a graph of the return force of the valve shown in FIG. 1 when installed on an inhaler and the contents dispensed through life.

### DETAILED DESCRIPTION

Metered dose inhalers in accordance with one aspect of the present invention include a metered dose valve comprising a helical spring made of a coil of elongate material, a seal, a seal support and a valve stem, the valve stem and seal being in mutual sliding sealing engagement, the helical spring exerting a force compressing the seal against the seal support, wherein the valve is configured and arranged such that a region of compressive contact is defined where a surface applying said force to the seal is substantially flat and extends in an arc through an angle in the range from about 180 to about 360 degrees. The seal onto which force is to be applied is typically an inner seal, i.e. the seal at the interior or inlet end of a metering chamber, also known as the metering gasket seal or tank seal.

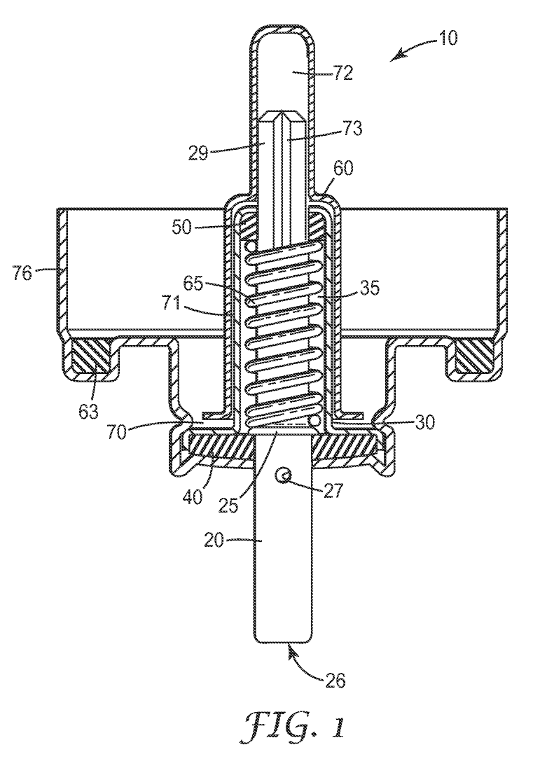

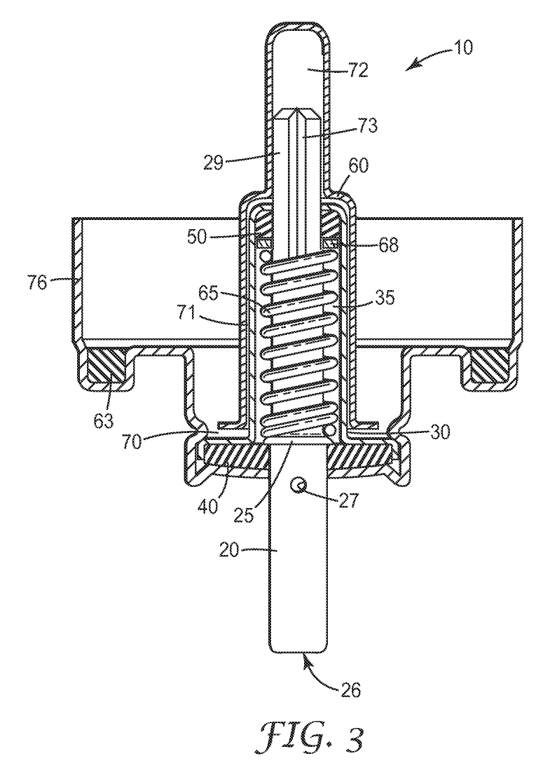

In certain embodiments of such inhalers, the valve is designed such that a washer is positioned between the helical spring and the seal so that the helical spring acts against a washer that in turn acts against the seal. This is best understood by reference to FIG. 3 showing a partial cross-sectional view of an exemplary valve (10) that is used with such an inhaler, the valve including such a washer. As can be seen in FIG. 3, a washer (68) is positioned between the helical compression spring (65) and an inner seal (i.e. the metering gasket seal) (50). (For a complete description of the valve, reference is made to the description in conjunction with FIG. 1, where similar parts are labeled with the same reference numbers.) As can be appreciated from FIG. 3, the helical spring may conveniently be a conventional helical spring, i.e. a spring made of a coil of elongate material where the elongate material in its cross-section is round along its length. Favorably the washer is made of a rigid material e.g. stainless steel, so as to provide a region of compressive contact when assembled into a valve The washer may be continuous in its perimeter or, desirably for ease in manufacture and/or assembly of the valve the washer may be a split-washer extending in an arc through angle in the range from about 270 up to but not including 360 degrees. Suitable washers include those having a rectangular cross section or, if appropriate depending on the particular valve design, having a dished-surface. The washer may have a vertical rim extending from the circumference to provide some lateral containment for the seal. To accommodate a washer into a valve previously designed without one, desirably the washer is thin (e.g. having a thickness of at most 1 mm, typically of about 0.25 mm).

Preferred embodiments of metered dose inhalers in accordance with one aspect of the present invention and metered dose valves in accordance with another aspect of the present invention, include a helical spring having an end portion facing the seal, said end portion of the helical spring having a substantially flat surface extending from the corresponding terminus of the coil of elongate material in an arc through an angle in the range from about 180 to about 360 degrees.

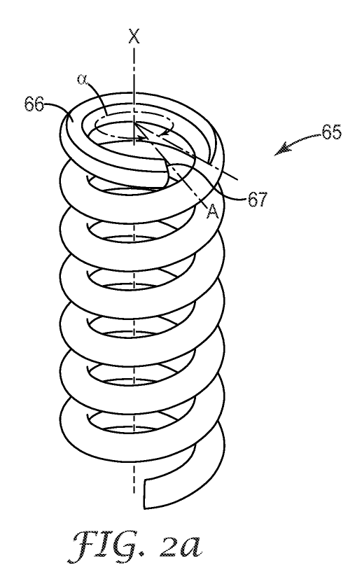

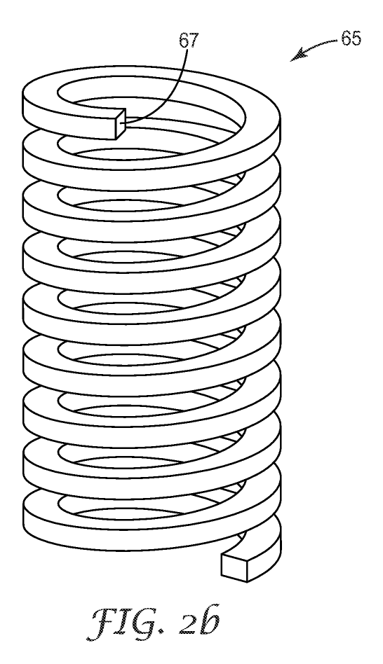

Reference is made here to FIG. 2a showing an isometric view of an exemplary compression spring (65) made of a coil of elongate material suitable for use in inhalers or valves described herein. A datum line, A, is normal to a longitudinal axis, X, of the spring and tangential to a terminus (67) of the coil of elongate material. A second line generally normal to the axis X and intersecting line A at said axis (to provide a locus point) sweeps out an angle, α, along the end portion of the spring, where the end portion of the spring has a substantially flat surface (66) extending from the terminus of the elongate material in an arc defined by angle α. In the exemplary spring shown in FIG. 2a, the helical spring has a substantially flat surface extending from the corresponding terminus of the coil of elongate material in an arc through an angle of about 330 degrees.

In embodiments described herein, where the end portion (facing the seal) of the helical spring of the valve has a substantially flat surface extending from the corresponding terminus of the coil of elongate material, said surface extends in an arc through an angle in the range from about 180 to about 360 degrees. In other words, the surface extends at least to an angle of about 180 degrees. Favorably, the surface extends at least to an angle of about 210 degrees, even more favorably at least about 240 degrees, yet even more favorably at least about 270 degrees, yet even more favorably at least about 300 degrees and most favorably at least about 330 degrees. As can be appreciated from FIG. 2a, the end portion of the spring is typically one turn of the coil, so that in embodiments where the complete end portion has a substantially flat surface, the relative angle would be then 360 degrees. Moreover any substantially flat surface beyond 360 degrees is typically no longer part of the end portion of the helical spring. In embodiments where the substantially flat surface is made in a post-treatment step (e.g. grinding) after the coil of elongate material has be formed into the helical spring, angles of 350 degrees or less (within the aforesaid favorably ranges) is typically favorable in terms of ease of such post-treatment and provision of desired valve performance using said spring.

Generally, the substantially flat surface of said end portion is approximately normal to the axis of the helical spring. It may be sloped slightly, depending on the particular form of the spring at rest, where its slope may depend on the desired or needed degree of compression of the spring during actuation. However if there is a slope in the end portion desirably it is much less than that of the pitch of the main body of the spring, for example, where the last coils (e.g. on each end) of the spring are made to have a shallower pitch than the remainder or main body of the spring.

In embodiments where the end portion of the spring has a substantially flat surface as described herein, it is favorable that the helical spring acts directly on the seal, It may be joined to a component that acts against the seal. However, it is preferred for the helical spring to act directly on the seal, since that obviates the need to either join another component or ensure correct assembly of another component, which would complicate the manufacturing process and may introduce variation in the metering volume.

For any embodiment described herein, the seal support may be any component that is static in relation to the valve as a whole, and provides the reaction force to the load applied by the spring on the seal. It is advantageously designed to control or direct the deformation of the seal upon such loading. The seal support may be formed at least in part by a valve body, such as a metering tank, or alternatively by an additional component interspersed between the seal and the housing, designed to control or direct the deformation of the seal upon loading.

For any embodiment described herein, the seal may be any shape to match the shape of the valve stem, including shapes described in U.S. Pat. No. 5,772,085 FIG. 2, herein incorporated by reference. Typically it will be annular and preferably in the form of an ‘O’ ring or an annulus with square or rectangular cross section.

For any embodiment described herein, the helical spring may be made of any suitable material with low creep. Typically, the material will have a Young's modulus that permits spring design with forces of around 30 Newtons when compressed to extents usual in aerosol valves e.g. 1 to 2 mm. Certain plastics materials may be appropriate to achieve advantageous shapes of spring, although metal, e.g. stainless steel, is the preferred material.

In favorable embodiments, the helical spring may be made from a coil of elongate material where the elongate material in its cross-section is rectangular along its length (e.g. as shown in FIG. 2b). In this way the spring provides a substantially flat surface on its end portion.

In other favorable embodiments the end portion of the helical spring may be provided with a substantially flat surface on its end portion, by stamping, crimping or end grinding step to provide the flat surface. Such processes would desirably provide a smooth substantially flat surface, which is close to orthogonal to the axis of the spring so that its load may be distributed as evenly as possible over the surface of the seal. When it is made by a process including grinding, this may include one of various end grinding processes known in the art. In one such process, springs are mounted in a rotary table by feeding them into through-holes circumferentially arranged in the table, and retaining them there with bushes but so that they protrude from the top and bottom faces of the table. A pair of superimposed and spaced grinding wheels is positioned to intersect the table above and below it so that, as the springs traverse the region of intersection, the tops and bottoms of the springs are ground by respective abrasive faces of the grinding wheels. The springs traverse substantially the whole faces of the grinding wheels during transit through the intersecting region. Where the springs enter the intersecting region, there is a lead-in region on the perimeters of the grinding wheels, which causes each spring to compress slightly before reaching the abrasive surfaces. It should be appreciated that the slight compression of the spring makes the pitch shallower, and hence the pitch of an end coil which already may have been formed shallower than the remainder of the spring. This allows a flat profile to be achieved which extends appreciably round the end coil without taking too much stock from an end of the flat profile nearest the end of the spring. Desirably stock is only removed up to, but not beyond the widest part of the elongate material from which the spring is made, so as to avoid the production of sharp edges and/or maintain smooth, in particular rounded, edges on the elongate material. Once ground, the springs may be poked out of the through-holes by rods operated by cams. However, to achieve the desired grinding the springs may be fed through a second time e.g. by reinserting into the circumferentially arranged through-holes. Moreover, although such springs for use in certain embodiments described herein only require to be flattened at one end, grinding at both ends obviates the need to orientate the springs for assembly into valves.

FIG. 1 provides a partial cross-sectional view of an exemplary metered dose dispensing valve (10). In use, the valve is crimped onto an aerosol container (not shown) via a ferrule (76), and a gasket seal (63) is provided to ensure a gas tight seal. Referring to FIG. 1, the valve (10) typically comprises a valve stem (20) that generally defines a longitudinal axis and a valve body (30), where the valve stem extends through a central aperture of the valve body. A lower stem portion (25) of the valve stem extends outwardly and is in slidable, sealing engagement with an outer seal (40) (also termed the diaphragm seal), while an upper stem portion (29) of the valve stem extends inwardly and is in slidable, sealing engagement with an inner seal (50) (also termed the metering gasket seal). A (non-transistory) metering chamber (35) is defined within the valve housing (30) between the outer seal (40) and inner seal (50). A compression spring (65) of the type shown in FIG. 2a is positioned within the valve housing with one end abutting the inner seal (50) and the other end abutting a flange (77) on the valve stem near the outer seal. As will be appreciated by the skilled reader, upon movement of the valve stem inwardly, a groove (73) in the upper stem portion (29) will pass beyond the inner seal (50) so that a complete seal is formed between the upper stem portion of the valve stem and the inner seal, thereby sealing off the metering chamber. Upon further movement of the valve stem inwardly, an opening (27) of the outlet passage of the valve stem passes the outer seal into the metering chamber and the contents of the metering chamber pass through the outlet passage of the valve stem, exiting the stem outlet. Referring to FIG. 1, the valve may comprise a second valve body (60) defining a bottle emptier. When such a second valve body is provided, aerosol formulation in the aerosol container (not shown) will pass through a gap (70) between the first valve body (30) and the second valve body (60) (the gap is near the outlet seal), through an annular gap (71) into a pre-metering region (72) and then through the groove (73) into the metering chamber (35). FIG. 2a shows the compression spring used in the exemplary valve shown in FIG. 1, which as described above, comprises a coil of elongate material (e.g. rounded stainless steel wire) about an axis, X, provided (e.g. through a end grinding process) with a substantially flat surface (66) extending from a terminus (67) of the coil through an angle, α, as indicated.

FIG. 2b shows an alternative compression spring (65) suitable for use in a metering valve or in a metering valve of an inhaler in accordance with the invention. The spring comprises a coil of elongate material that is rectangular in its cross section with thus a rectangular terminus (67) and a flat surface extending over the complete end portion of the spring and hence through an angle, α, of 360 degrees.

Metered dose inhalers in accordance with one aspect of the present invention comprise an aerosol container fitted with a metered dose aerosol valve as described herein, said container containing a medicinal formulation comprising a medicament and HFA 134a and/or HFA 227 and being substantially free of ethanol and substantially free of surfactant.

Metered dose valves in accordance with the second aspect of the present invention comprising a helical spring having an end portion facing the seal, where said end portion of the helical spring having a substantially flat surface extending from the corresponding terminus of the coil of the elongate material in an arc through an angle in the range 180 to 360 degrees may be used to dispense any medicinal aerosol formulation, for example a formulation used in a pressurized metered dose inhaler. However such valves are particularly advantageous for use with metered dose inhalers for dispensing medicinal aerosol formulations comprising a medicament and HFA 134a and/or HFA 227 and being substantially free of ethanol and substantially free of surfactant.

Metered dose inhalers and valves described herein are particular advantageous for dispensing such medicinal aerosol formulations that are free of surfactant. Alternatively or additionally metered dose inhalers and valves described herein are particular advantageous for dispensing such medicinal aerosol formulations that are free of ethanol.

Medicinal aerosol formulations may contain a medicament dispersed therein or a medicament in solution or alternatively a combination of two or more medicaments (where one medicament is dispersed, another is in solution or where all medicaments are in solution or where all medicaments are dispersed). Metered dose inhalers and valves described herein are particularly suitable for dispensing such medicinal aerosol formulations comprising a medicament (e.g. at least one medicament) dispersed in said formulation.

Medicament may be a drug, vaccine, DNA fragment, hormone or other treatment. The amount of medicament would be determined by the required dose per puff and available valve sizes, which are typically 25, 50 or 63 microlitres, but may include 100 microlitres where particularly large doses are required.

Suitable drugs include those for the treatment of respiratory disorders, e.g., bronchodilators, anti-inflammatories (e.g. corticosteroids), anti-allergics, anti-asthmatics, anti-histamines, and anti-cholinergic agents. Therapeutic proteins and peptides may also be employed for delivery by inhalation.

Exemplary drugs which may be employed for delivery by inhalation include but are not limited to: albuterol, terbutaline, ipratropium, oxitropium, tiotropium, beclomethasone, flunisolide, budesonide, mometasone, ciclesonide, cromolyn sodium, nedocromil sodium, ketotifen, azelastine, ergotamine, cyclosporine, salmeterol, fluticasone, formoterol, procaterol, indacaterol, TA2005, omalizumab, zileuton, insulin, pentamidine, calcitonin, leuprolide, alpha-1-antitrypsin, interferons, triamcinolone, and pharmaceutically acceptable salts and esters thereof such as albuterol sulfate, formoterol fumarate, salmeterol xinafoate, beclomethasone dipropionate, triamcinolone acetonide, fluticasone propionate, tiotropium bromide, leuprolide acetate and mometasone furoate.

Medicinal aerosol formulations may comprise excipients other than surfactants or ethanol, such as a particulate bulking agent, which may be in micronized or submicron form. Examples of such include lactose, sucrose, alanine, sodium sulphate. Other propellant components are possible, such as gases or liquids soluble in the HFAs e.g. CO₂.

### EXAMPLES

### Example 1

Metering valves of the type shown in FIG. 1 and including a helical spring having a form as shown in FIG. 2a, with metering volume nominally 63 microlitres, were assembled. The helical spring was a right-handed helical spring of length 11.6 mm and external diameter approximately 4.8 mm, made from Stainless steel grade 302S26 Grade II round wire of diameter 0.66 mm. The pitch on the last 1½ turns was shallower than that of the other 5 turns. In its uncompressed state the average pitch of the spring was 6.6 degrees. The spring had a flat profile on both of its end portions, extending through an angle, α, of 330 degrees from each terminus of the wire, provided by a grinding process.

Aerosol 10 ml canisters were cold-filled with a suspension formulation containing 1.97 mg/ml micronized albuterol sulfate in HFA 134a, and the valves crimped in place.

### Example 2

Metering valves as specified in Example 1, with a single difference in that the round wire had diameter of a 0.74 mm (the helical spring still had an external diameter of approximately 4.8 mm), were assembled. Aerosol 10 ml canisters were cold-filled with a suspension formulation containing 1.97 mg/ml micronized albuterol sulfate in HFA 134a, and the valves crimped in place.

### Comparative Example

Metering valves as specified in Example 1 were assembled, but with a helical spring differing in its form. Here the helical spring was provided with a flat profile on both end portions of the spring, extending through an angle of 90 degrees from each terminus of the wire, by grinding. As in the Examples 1 and 2, aerosol 10 ml canisters were cold-filled with a suspension formulation containing 1.97 mg/ml micronized albuterol sulfate in HFA 134a, and the valves crimped in place.

### Methodology

Following a period of 7 days for acclimatization of the seals in the aerosol units they were tested for the force characteristics of the valve after actuating various numbers of doses through the life of the unit.

The sampling regime was as follows:
1. Insert the aerosol unit into a fresh actuator, and prime the inhaler, i.e. shake the inhaler with a gentle rocking action through 180° inversion for at least 10 seconds and immediately fire two shots to waste.
2. Weigh the aerosol unit with actuator, then fire one shot. Repeat five times, followed by a further weighing, and calculate five shot weights by subtracting weighings.
3. Fire two priming shots. Insert aerosol unit into a tensile tester and record the profile of force against valve travel for firing and return of the valve at 20 mm/min. Record the Force to fire the valve, corresponding to the point in the cycle where the stem side hole first breaks through the diaphragm seal during its inward travel. Record the Return Force for the valve, corresponding to the point in the cycle where the stem groove first breaks through the tank seal during its outward travel.
4. Repeat step 3, two more times.
5. Fire 40 shots to waste using an automatic valve firing machine.
6. Repeat steps 1 to 5.
7. Repeat steps 1 to 4, then fire 40 shots to waste using an automatic valve firing machine.
8. Repeat steps 1 to 4.

### Results

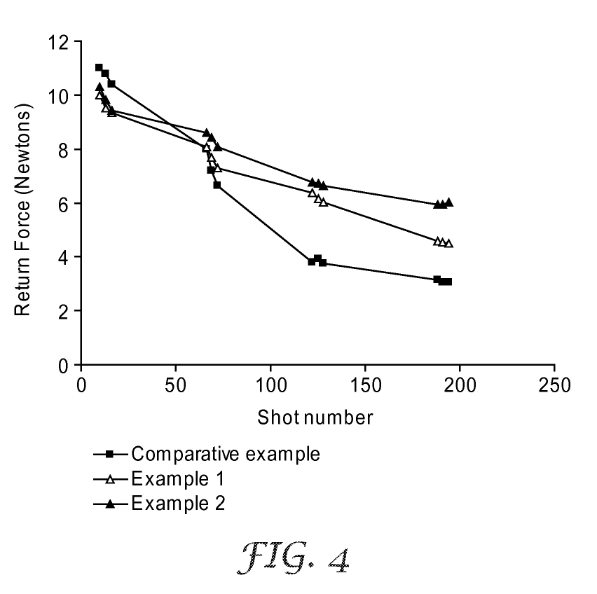

The return force through life (mean of readings for 5 separate inhaler units) for examples 1 and 2 and the comparative example is shown in FIG. 4 as a function of number of shots dispensed from the inhaler. Relative to the comparative example, Examples 1 and 2 show a significant and effective reduction in the decrease of return force over the lifetime of the inhalers. The significance of this difference is demonstrated in Table 1, which compares the occurrences of zero-return-force (Zero RF) and significantly reduced shot weight, i.e. a low shot weight less than 67 mg, for the examples and the comparative example. Moreover Examples 1 and 2 showed no occurrence of a zero-return-force, nor any occurrence of a shot weight of less than 67 mg over the lifetime of the tested inhalers.

TABLE 1

| | | | Low shot* | Low shot* |
|---|---|---|---|---|
| | Zero RFs (5 | Zero RFs (5 | weight (5 | weight (5 |
| | units: shots | units: shots | units: shots | units: shots |
| | 122, 125, 128) | 188, 191, 194) | 115-119) | 181-185) |
| Compar- | 4 | 6 | 10 | 7 |
| ative | | | | |
| example | | | | |
| Example 1 | 0 | 0 | 0 | 0 |
| Example 2 | 0 | 0 | 0 | 0 |

*A low shot is less than 67 mg. Other shots average 76 mg.

It is to be understood that the invention is not limited to exemplary embodiments explicitly described herein, in particular in aforesaid example section, but extends to other embodiments that the skilled person would contemplate based on the general teaching herein

1-27. (canceled)

28. A metered dose inhaler comprising an aerosol container fitted with a metered dose aerosol valve and containing a medicinal formulation, the formulation comprising a medicament and HFA 134a and/or HFA 227 and being substantially free of ethanol and substantially free of surfactant, and
the valve comprising a helical spring, a seal, a seal support and a valve stem, the valve stem and seal being in mutual sliding sealing engagement, the helical spring comprising a coil of elongate material and exerting a force compressing the seal against the seal support wherein the valve is configured and arranged such that a region of compressive contact is defined where a surface applying said force to the seal is substantially flat and extends in an arc through an angle in the range from about 180 to about 360 degrees.

29. A metered dose inhaler according to claim 28 comprising a split-washer extending in an arc through angle in the range from about 270 up to but not including 360 degrees or a washer, said split-washer or washer being positioned between the helical spring and the seal.

30. A metered dose inhaler according to claim 28 where the helical spring has an end portion facing the seal, said end portion of the helical spring having a substantially flat surface extending from the corresponding terminus of the coil of elongate material in an arc through an angle in the range from about 180 to about 360 degrees.

31. A metered dose inhaler according to claim 28 in which the elongate material comprises a flat surface along its entire length.

32. A metered dose inhaler according to claim 28 in which the helical spring acts directly on the seal.

33. A metered dose aerosol valve comprising a helical spring, a seal, a seal support and a valve stem, the valve stem and seal being in mutual sliding sealing engagement, the helical spring comprising a coil of elongate material and exerting a force compressing the seal against the seal support, wherein the helical spring has an end portion facing the seal, said end portion of the helical spring having a substantially flat surface extending from the corresponding terminus of the coil of the elongate material in an arc through an angle in the range about 180 to about 360 degrees.

34. A metered dose inhaler comprising an aerosol container fitted with a metered dose aerosol valve according to claim 33 for dispensing a medicinal formulation comprising a medicament and HFA 134a and/or HFA 227 and being substantially free of ethanol and substantially free of surfactant.

35. A metered dose inhaler according to claim 28 or 34, wherein said medicinal formulation is free of surfactant.

36. A metered dose inhaler according to claim 28 or 34, wherein said medicinal formulation is free of ethanol.

37. A metered dose inhaler according to claim 28 or 34, wherein said medicinal formulation comprises a medicament that is dispersed said formulation.

38. A metered dose inhaler according to claim 28 or 34, wherein said medicinal formulation comprises a medicament selected from the group consisting of albuterol, terbutaline, ipratropium, oxitropium, tiotropium, beclomethasone, flunisolide, budesonide, mometasone, ciclesonide, cromolyn sodium, nedocromil sodium, ketotifen, azelastine, ergotamine, cyclosporine, salmeterol, fluticasone, formoterol, procaterol, indacaterol, TA2005, omalizumab, zileuton, insulin, pentamidine, calcitonin, leuprolide, alpha-1-antitryp sin, interferon, triamcinolone, and pharmaceutically acceptable salts and esters thereof and mixtures thereof.
